(19) United States
(12) Patent Application Publication
(10) Pub. No.: US 2024/0091669 A1
(43) Pub. Date: Mar. 21, 2024

(54) SEMI-AUTOMATIC CRYSTALLIZATION PROCESS TESTING

(71) Applicant: Saudi Arabian Oil Company, Dhahran (SA)

(72) Inventors: Faisal Alrasheed, Dammam (SA); Fahd Ibrahim AlGhunaimi, Dhahran (SA); Tao Chen, Dhahran (SA)

(21) Appl. No.: 17/933,763

(22) Filed: Sep. 20, 2022

(51) Int. Cl. B01D 11/02; G01N 33/18

(52) U.S. Cl. CPC B01D 11/0203; B01D 11/0207; G01N 33/1813; G01N 33/182; G01N 2030/009

(57) ABSTRACT

A method for using an accelerated solvent extractor to study ion reaction parameters is provided. The method includes entering a solution mixture and reaction parameters for each of a plurality of extraction cells, wherein the solution mixture includes a ratio of a first ion solution to a second ion solution. The solution mixture is reacted in each of the plurality of extraction cells at the reaction parameters for that extraction cell. Effluent and solids are collected from each of the plurality of extraction cells. The effluent and the solids are analyzed to obtain composition results. The composition results are correlated to the solution mixture for each of the plurality of extraction cells and the reaction parameters for each of the plurality of extraction cells.

### TECHNICAL FIELD

The present disclosure is directed to a method for using an accelerated solvent extractor as a crystallization tester.

### BACKGROUND

Crystallization is a chemical process that can be used for chemical production or purification across numerous industries. Crystallization is the method of choice to produce materials including sodium chloride, sodium sulfate, aluminum sulfates, and many others. Furthermore, crystallization reduces the energy needed for purification of substances because the enthalpies of crystallizations are less than the enthalpies of vaporization. Crystallization reactions can also be used for waste mitigation, such as removing ions from mining wastes.

Seawater injection is one of the methods used in the oil and gas industry to maintain pressure in reservoirs. The injection increases recovery and can help to maintain production rates of the reservoirs. However, seawater can contain large quantities of sulfate, which can contribute to scaling and may be incompatible with the reservoir rocks and formation fluids. Scale formation, including that of sulfate-based scales, is a major problem in oil and gas production and processing facilities. It reduces through put by inhibiting or restricting the hydrocarbon flow pathways. In addition, it affects water production and transportation. Mineral Scale formation can cause major challenges in flow assurance, formation damage, reduction of flow in production and injection, and equipment damage. This can increase the need for workovers and remediation. Further, sulfate can be used by sulfate reducing bacteria, which metabolize it to form hydrogen sulfide.

### SUMMARY

An embodiment described herein provides a method for using an accelerated solvent extractor to study ion reaction parameters. The method includes loading a first solvent vessel with a first ion solution, loading a second solvent vessel with a second ion solution, and entering a solution mixture for each of a plurality of extraction cells, wherein the solution mixture includes a ratio of the first ion solution to the second ion solution. The method further includes entering reaction parameters for each of the plurality of extraction cells. The solution mixture is reacted in each of the plurality of extraction cells at the reaction parameters for that extraction cell. Effluent is collected from each of the plurality of extraction cells in each of a plurality of collection vials. Solids are collected from each of the plurality of extraction cells. The effluent and the solids are analyzed for each of the plurality of extraction cells to obtain composition results. The composition results for the effluent and the solids for each of the plurality of extraction cells are correlated to the solution mixture for each of the plurality of extraction cells and the reaction parameters for each of the plurality of extraction cells.

### BRIEF DESCRIPTION OF DRAWINGS

FIG. 1 is a simplified process flow diagram of an accelerated solvent extractor (ASE).

FIG. 2A is a drawing of an extraction cell used in an accelerated solvent extractor.

FIG. 2B is a transparent view of the extraction cell 120 showing the packing of materials in the extraction cell.

FIG. 3 is a drawing of an accelerated solvent extractor (ASC) 300 that may be used in embodiments described herein.

FIG. 4 is a process flow diagram of a method for using an accelerated solvent extractor to study the parameters affecting a crystallization process.

### DETAILED DESCRIPTION

Precipitation of sulfate is one of the cost-effective methodologies to remove sulfate from seawater. For example, barium chloride can react with sulfate from seawater to form barium sulfate crystals, which are insoluble in water. Studying this reaction to optimize the crystallization process is important to determine the rates of nuclei formation and crystal growth in addition to other factors. The techniques described herein utilize an accelerated solvent extractor (ASE) as a test platform to optimize the crystallization process parameters. This can be used to achieve desired product specifications in terms of rate of nuclei formation and crystal growth.

The accelerated solvent extraction (ASE), which is also called pressurized fluid extraction (PFE) or pressurized liquid extraction (PLE), is a methods of sample preparation. ASE uses a liquid solvent extraction technique in which aqueous and organic solvents are used to extract materials from solids at wide ranges of temperature and pressure. The temperature can be ranged from ambient temperature to 200° C. Similarly, the pressure can range from atmospheric to 1500 PSI. With modifications, the process may be used at higher temperatures and pressures, or at lower temperatures and pressure.

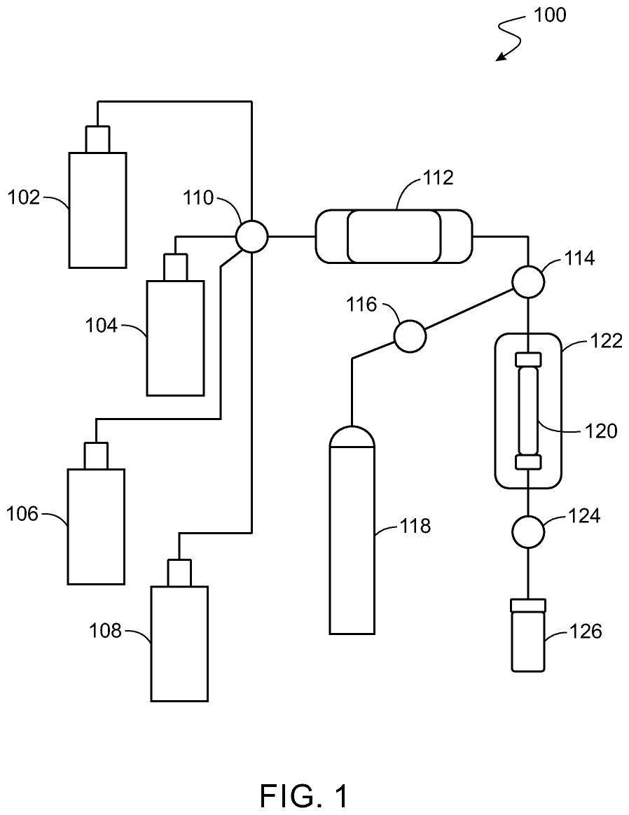

FIG. 1 is a simplified process flow diagram of an accelerated solvent extractor (ASE) 100. The ASE 100 has a number of solvent vessels 102, 104, 106, and 108 that can hold reactants, solvents, rinse solutions, and the like. Although the solvent vessels 102-108 are generally liquid vessels, in some embodiments, a gas vessel is used to hold a gaseous reactant instead of, or in addition to, one of the solvent vessels.

The solvent vessels 102-108 are coupled to a selector valve 110, which selects which solvent vessel is being used for the liquid feed. In some embodiments, the selector valve 110 allows for the mixing of liquids from different solvent vessels, such as allowing a reagent added to the first solvent vessel 102 to be diluted, for example, by a rinse solvent in a second solvent vessel 104.

The liquid feed passes into a liquid pump 112 to be fed to the rest of the ASE 100. An addition valve 114 selects between the liquid feed and a gas feed from a purge valve 116 that is fluidically coupled to an inert gas cylinder 118, such as a nitrogen cylinder. The nitrogen gas is used for purging lines and equipment. In some embodiments, the addition valve 114 is fluidically coupled to other gas cylinders (not shown), for example, to add a gaseous feed such as light hydrocarbons. Further, in some embodiments, the addition valve 114 is used to fluidically couple a vacuum pump to the system upstream of the extraction cell 120.

The liquid or gaseous feeds are sent to the extraction cell 120 for reaction. The temperature of the extraction cell 120 is controlled by a temperature jacket 122. Some compounds will crystalize better at higher temperatures, such as calcium sulfate. Other compounds, such as clathrate hydrates, will crystalize better at lower temperatures. Accordingly, in some embodiments, the temperature jacket 122 is an oven that is capable of heating the extraction cell 120 to about 50° C., 100° C., 200° C., or higher. In some embodiments, the oven heats the extraction cell to between about 100° C. and about 200° C. The temperature jacket 122 is not limited to an oven. In some embodiments, the temperature jacket 122 can be a cooler used to cool the extraction cell 120, for example, to 10° C., −10° C., −50° C., or lower.

A static valve 124 at the outlet of the extraction cell 120 allows the extraction cell 120 to be blocked in during the preset reaction time. Once the reaction time is over, the static valve 124 is opened to allow flow through the extraction cell 120 to a collection vial 126.

The liquid feeds from the four solvent reservoirs 102-108 can be mixed at different ratios, to allow for control over polarity, dielectric constants, and stoichiometric ratios. This provides control of the concentration of each of the reactant, which may be used with a particle size analyzer at the outlet of the extraction cell 120. In some embodiments, water is used as one of the solvents, for example, to dilute reactants. In some embodiments, the ASE 100 operates up to 24 extraction cells, wherein the reaction and crystallization parameters for each extraction cell 120 are individually set through a control panel or using a program on a computer interfaced with the ASE-100. After entry of the parameters, the operation of the ASE 100 is unattended.

The techniques are used to add several reactants in series or as mixtures to each extraction cell 120. The extraction cells are then used as reactors, wherein the reaction parameters for each extraction cell 120 are varied. In various embodiments, the reaction parameters include temperature, pressure, residence time, reactant ratios, concentrations, or catalysts, among others. Accordingly, the techniques can be used to optimize, test, and carry out various reactions, such as crystallizations and purifications.

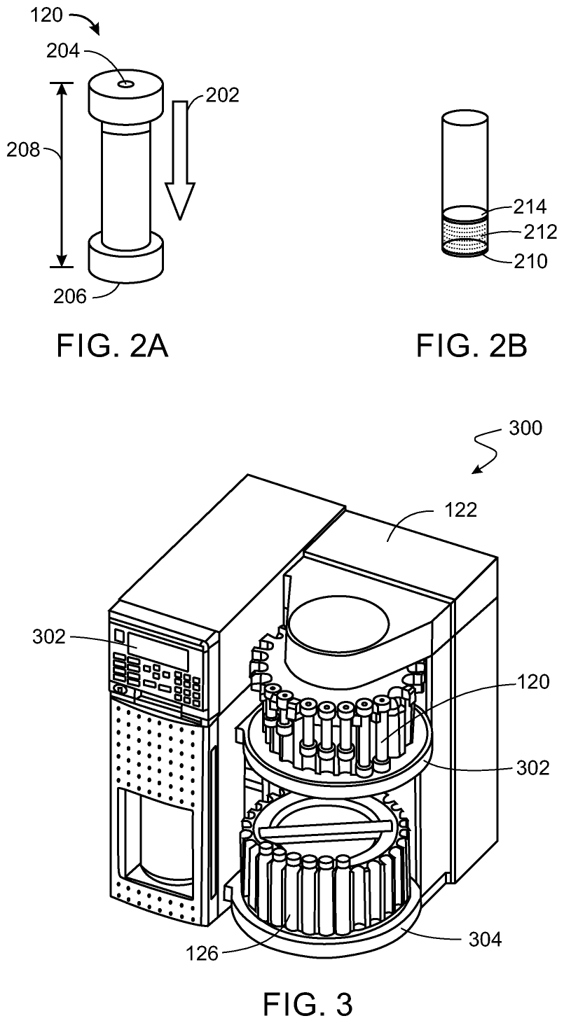

FIG. 2A is a drawing of an extraction cell 120 used in an accelerated solvent extractor. The extraction cell 120 may be made from stainless steel, nickel, or corrosion resistant alloys, such as Hastelloy. The flow 202 of materials through the extraction cell 120 proceeds from a top opening 204 to a bottom opening 206. The volume of the extraction cell 120 is determined by its length 208. For example, and extraction cell 120 with a length of about 4 cm may have a volume of about 0.75 mL. A shorter extraction cell 120 will have a proportionally smaller volume. The volume of different extraction cells may be selected for different reactions, or a mixture of different volumes may be used in a single reaction. Further, the volume of the extraction cells may affect the reaction time, with smaller extraction cells having lower reaction times.

FIG. 2B is a transparent view of the extraction cell 120 showing the packing of materials in the extraction cell 120. In some embodiments, a filter 210 is placed at the bottom of the extraction cell 120, for example, over the bottom opening 206 using a filter insertion tool. In various embodiments, the filter 210 is formed from cellulose, porous ceramic, or porous stainless steel. For the porous ceramic or porous stainless steel, the pore sizes of the filter 210 may be selected based on the expected size of solids formed during the reaction, such as pores of up to about 100 μm in size, up to about 50 μm in size, up to about 10 μm in size, or smaller. Smaller pore sizes will increase the pressure used to filter solids from the reactant, while larger pore sizes will increase the risk of losing solids to the collection vial.

The extraction cell 120 is not limited to a single filter, but may have multiple filters or filter materials added. For example, a filter material 212 may be added to the extraction cell 120 over the filter 210. The filter material 212 may be an adsorbent used to capture certain reactants, providing a separation of soluble reactants, potentially increasing the accuracy of the analysis. A second filter material 214 can be placed over the filter material 210 to capture solids formed in the reaction, preventing them from mixing with the filter material 212.

FIG. 3 is a drawing of an accelerated solvent extractor (ASC) 300 that may be used in embodiments described herein. Like numbered items are as described with respect to FIG. 1. This ASE 300 provides a number of extraction cells 120 and collection vials 126, and is available from ThermoFisher Scientific, of Waltham, MA, USA, for example, under the Dionex ASE line of products.

The ASE 300 provides the pump, oven, a tray 302 to hold the extraction cells 120, and a tray 304 to hold the collection vials 126. The ASE 300 can be modified with different metallurgy, such as hastelloy, to improve the various chemical compatibility. This may include changing the extraction cell 120, the pump, tubing, and solvent reservoirs. The pump can be modified with a back-flush or other self-cleaning mechanism to lower the probability of cross-contamination of reactants.

In the example shown in FIG. 3, a control panel is used to enter parameters. In some embodiments, a computer is interfaced to the ASE 300 to enter the parameters. Parameters of an example of the dissolution are presented in Table 1. All the parameters can be controlled via a computer or via the instrument screen. A nitrogen tank, not shown, is fluidically coupled to the ASE 300 to provide a nitrogen gas (99.95% purity) for purging. The parameters of the ASE 300 can be modified to fit the extraction, pretreatment, and purify as per the preference of the operators.

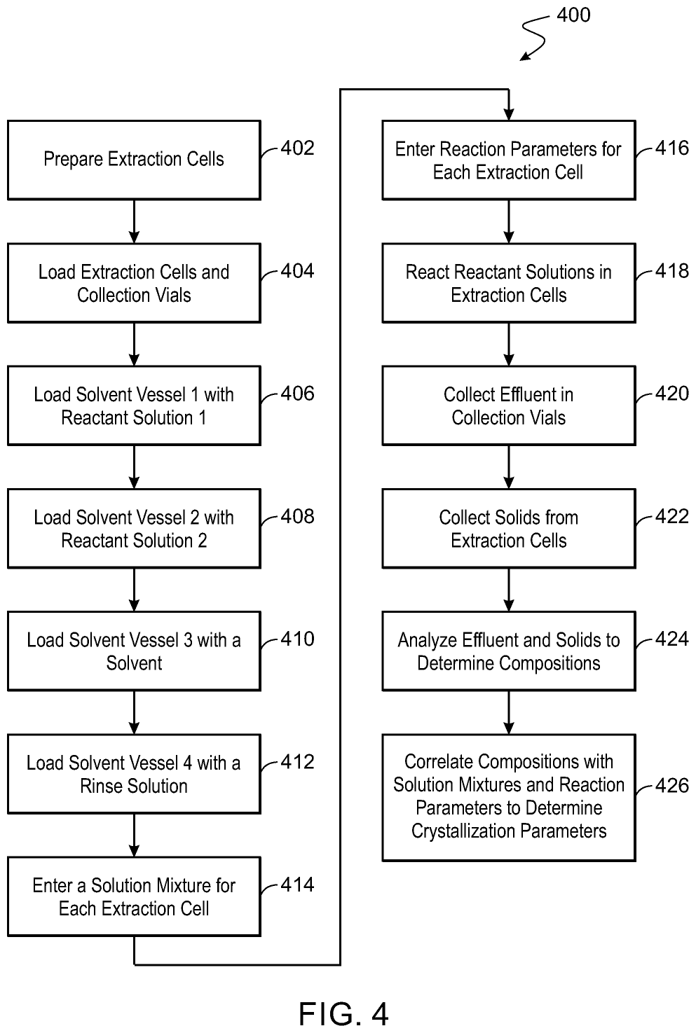

FIG. 4 is a process flow diagram of a method 400 for using an accelerated solvent extractor to study the parameters affecting a crystallization process. The method 400 begins at block 402, with the preparation of the extraction cells. For example, a filter insertion tool is used to insert a filter at the bottom of each of the extraction cells. In some embodiments, an adsorption media is added to the extraction cells over the filter, and a second filter is placed over the adsorption media.

At block 404, the extraction cells are loaded into the ASE, for example, in an extraction cell tray, which correlates the location of the extraction cells to numbered entries in a parameter table. Collection vials are loaded into the ASE, for example, in a collection vial tray, which correlates the location of the collection vials to numbered entries in the parameter table.

At block 406, solvent vessel 1 is loaded with reactant solution 1. At block 408, solvent vessel 2 is loaded with reactant solution 2. At block 410, solvent vessel 3 is loaded with a solvent. At block 412, solvent vessel 4 is loaded with a rinse solution, such as water. The solutions are not limited to the descriptions above, but may be changed to match the specific reaction parameters. For example, solvent vessel 3 may be loaded with another reactant, a strong acid, a strong base, or other materials depending on the reaction that is to be studied. Any number of other changes may be made based on the reaction to be studied. Some chemical reactants require the adjustment of the metallurgy for compatibility.

At block 414, a solution mixture for each extraction cell is entered. As described herein, this may be performed through the control panel of the ASE, or through a computer attached to an interface of the ASE. The reactants can be added to any extraction cell in parallel or in series. They can be also premixed in the reservoir, for example, diluted for concentration by water for example.

At block 416, reaction parameters for each extraction cell are entered. As described herein, the reaction parameters may include the soaking time, flush %, static time, pressure, temperature, solvent type, or acid type and ratio, preheat time, purge, residence time, ratio of the reactants, concentration, and cycle, among others. Further, the operator can add a particle size controller to control the reaction.

At block 418, the reactant solutions are reacted in the extraction cells. This may be performed statically, by closing the static valve at the base of the extraction cell, or dynamically, by flowing the reactants through the extraction cell, collecting any solids formed on the filter, and capturing the effluent in the corresponding collection vial.

At block 420, the effluent from the extraction cell is collected in the collection vial. For example, if the reaction was performed statically, the static valve is opened to allow fluid to flow from the extraction cell. In some embodiments, a rinse solution may be used to wash excess reactants from the extraction cell to the collection cell. In various embodiments, the contents of the extraction cell may be pushed to the collection cell using the nitrogen.

At block 422, the solids are collected from the extraction cells. For example, this can be performed by removing the extraction cell from the ASE and blowing the contents out with a nitrogen stream applied to the bottom of the extraction cell.

At block 424, the effluent and the solids are analyzed to determine composition. For the effluent, this may be performed by ion chromatography, spectroscopy, flame ionization spectroscopy, or any number of other techniques. For the solids, this may be performed by x-ray diffraction (XRD), x-ray absorbance-fluorescence spectroscopy (XAFS), particle size analysis, or any number of other techniques.

At block 426, the composition of the effluent and the solids are correlated with the solution mixtures and the reaction parameters to determine the parameters that control the reaction. For example, the particle size may be correlated with the reaction temperature and pressure.

### Example

As a theoretical example of the operation, a seawater solution can be reacted with barium chloride in the extraction cell. Produced water from a reservoir can be used for the reaction, for example, being spiked with the barium chloride solution. Accordingly, in this example, reservoir 1 contains seawater, reservoir 2 contains the barium chloride solution, reservoir 3 contains with distilled water, and reservoir 4 contains a particle-size control chemical. Some materials that can be used to control the particle size include fibrous and waxy substances, or soft material. Other chemicals can be used to provide active nucleation sights or function as agglomeration agents. ASE parameters that may be used in a barium chloride test are summarized in Table 1.

TABLE 1
Method parameters for the reaction.

| Parameter | Reaction Conditions |
|---|---|
| Solvents | 1M barium chloride (solvent1) |
| (reactants) | Seawater (solvent2) |
| | Water (solvent 3) |
| Flush % | 50 |
| Static min | 50 |
| Pressure psi | 1000 |
| Temperature ° C. | 80 |
| Reaction | 30% seawater |
| | 70% barium chloride solution |
| Preheat min | 1 |
| Purge s | 20 |
| Cycle | 3 |

As described herein, the extraction cell is used as the reactor. The seawater, the barium chloride, or both can be diluted with water to vary the concentration. The barium chloride is reacted with the seawater. The product is washed with the particle size controller to stop the agglomeration. The values used to carry crystallization reactions can be varied based on the operations. The pressure can be increased when a backpressure valve or a check valve are applied. In addition, the cell can be upgraded with materials that can handle high pressure. The temperature can be varied up to 200° C. and increased when the oven is upgraded and the metallurgy of the other parts can handle the higher temperature.

In comparison to conventional reaction methods, the ASE 100 provides a number of advantages. The reactions in the extraction cells take substantially less time than other techniques, for example, taking about five minutes, or less, of preparation time per sample followed by automated operation at numerous preselected conditions. By comparison, conventional processes to test reaction kinetics often take hours to run. The total preparation time required is less than five minutes per sample (including the pre-treatment) for preparations, and then the ASE can perform up to 24 samples' separations in an unattended operation or multiple reaction conditions per each sample. The conventional method will take nearly half a day per reaction on average. This invention is more environmentally friendly, as there is less need to dispose of used solvents after finishing the experiment. In summary, the ASE protocol requires at least 50% less solvent, 95% less contact time, faster, and more robust. The technique is semi-automated.

### Embodiments

An embodiment described herein provides a method for using an accelerated solvent extractor to study ion reaction parameters. The method includes loading a first solvent vessel with a first ion solution, loading a second solvent vessel with a second ion solution, and entering a solution mixture for each of a plurality of extraction cells, wherein the solution mixture includes a ratio of the first ion solution to the second ion solution. The method further includes entering reaction parameters for each of the plurality of extraction cells. The solution mixture is reacted in each of the plurality of extraction cells at the reaction parameters for that extraction cell. Effluent is collected from each of the plurality of extraction cells in each of a plurality of collection vials. Solids are collected from each of the plurality of extraction cells. The effluent and the solids are analyzed for each of the plurality of extraction cells to obtain composition results. The composition results for the effluent and the solids for each of the plurality of extraction cells are correlated to the solution mixture for each of the plurality of extraction cells and the reaction parameters for each of the plurality of extraction cells.

In an aspect, the first ion solution includes seawater. In an aspect, the second ion solution includes barium chloride.

In an aspect, the method includes loading a third solvent vessel with a rinse solution. In an aspect, the rinse solution includes water.

In an aspect, the first ion solution, or the second ion solution, or both, is diluted before forming the solution mixture.

In an aspect, the reaction parameters include a reaction temperature for an extraction cell. In an aspect, the reaction temperature is between 100° C. and 200° C. In an aspect, the reaction temperature is between −100° C. and 20° C.

In an aspect, the reaction parameters include a reaction pressure for an extraction cell. In an aspect, the reaction pressure is between 1 atm (101 kPa) and 110 atm (11100 kPa).

In an aspect, the effluent includes unreacted liquid from the solution mixture.

In an aspect, the solids include crystals formed by the reaction of the first ion solution and the second ion solution. In an aspect, the solids include insoluble salts formed by the reaction of the first ion solution and the second ion solution.

In an aspect, the method includes loading a third solvent vessel with a modifier solution. In an aspect, the modifier solution includes a nucleating agent. In an aspect, the modifier solution includes a crystallite size control agent. In an aspect, the modifier solution includes an organic compound.

In an aspect, analyzing the effluent includes determining the concentration of ions.

In an aspect, analyzing the solids includes measuring properties of the solids with x-ray fluorescence spectrometry.

Other implementations are also within the scope of the following claims.

What is claimed is:

1. A method for using an accelerated solvent extractor to study ion reaction parameters, comprising:
loading a first solvent vessel with a first ion solution;
loading a second solvent vessel with a second ion solution;
entering a solution mixture for each of a plurality of extraction cells, wherein the solution mixture comprises a ratio of the first ion solution to the second ion solution;
entering reaction parameters for each of the plurality of extraction cells;
reacting the solution mixture in each of the plurality of extraction cells at the reaction parameters for that extraction cell;
collect effluent from each of the plurality of extraction cells in each of a plurality of collection vials;
collect solids from each of the plurality of extraction cells;
analyze the effluent and the solids for each of the plurality of extraction cells to obtain composition results; and
correlate the composition results for the effluent and the solids for each of the plurality of extraction cells to the solution mixture for each of the plurality of extraction cells and the reaction parameters for each of the plurality of extraction cells.

2. The method of claim 1, wherein the first ion solution comprises seawater.

3. The method of claim 1, wherein the second ion solution comprises barium chloride.

4. The method of claim 1, comprising loading a third solvent vessel with a rinse solution.

5. The method of claim 4, wherein the rinse solution comprises water.

6. The method of claim 5, wherein the first ion solution, or the second ion solution, or both, is diluted before forming the solution mixture.

7. The method of claim 1, wherein the reaction parameters comprise a reaction temperature for an extraction cell.

8. The method of claim 7, wherein the reaction temperature is between 100° C. and 200° C.

9. The method of claim 7, wherein the reaction temperature is between −100° C. and 20° C.

10. The method of claim 1, wherein the reaction parameters comprise a reaction pressure for an extraction cell.

11. The method of claim 1, wherein the reaction pressure is between 1 atm (101 kPa) and 110 atm (11100 kPa).

12. The method of claim 1, wherein the effluent comprises unreacted liquid from the solution mixture.

13. The method of claim 1, wherein the solids comprise crystals formed by the reaction of the first ion solution and the second ion solution.

14. The method of claim 1, wherein the solids comprise insoluble salts formed by the reaction of the first ion solution and the second ion solution.

15. The method of claim 1, comprising loading a third solvent vessel with a modifier solution.

16. The method of claim 15, wherein the modifier solution comprises a nucleating agent.

17. The method of claim 15, wherein the modifier solution comprises a crystallite size control agent.

18. The method of claim 15, wherein the modifier solution comprises an organic compound.

19. The method of claim 1, wherein analyzing the effluent comprises determining the concentration of ions.

20. The method of claim 1, wherein analyzing the solids comprises measuring properties of the solids with x-ray fluorescence spectrometry.
